(19) United States
(12) Patent Application Publication
(10) Pub. No.: US 2002/0111464 A1
(43) Pub. Date: Aug. 15, 2002

(54) Rapid diagnostic test for neospora caninum infections in animals

(76) Inventor: Gerhardt Schurig, Blacksburg, VA (US)

Correspondence Address: James W. Hiney, Esq., Suite 1100, 1872 Pratt Drive, Blacksburg, VA, 24060, US

(21) Appl. No.: 10/097,757

(22) Filed: Mar. 14, 2002

Related U.S. Application Data
(60) Provisional application No. 60/268,282, filed on Feb. 14, 2001.

(51) Int. Cl. C07K 1/00; C07K 14/00; C07K 17/00; A61K 9/00

(52) U.S. Cl. 530/350

(57) ABSTRACT

This invention relates to a kit for rapid detection of Neospora caninum infections in animals, especially cattle. The kit is based on a latex agglutination assay to carry out an on-site test which employs negative and positive control samples to identify said infections. Serum taken from the animal is mixed with prepared latex beads containing which is then incubated for a few minutes to detect clumping of the beads.

This patent application depends on and incorporates by reference an earlier filed provisional application Serial No. 60/268,282 of the same title which was filed on Apr. 25, 2001. The inventor of both this application, Gerhardt Schurig, and the provisional application is the same and a claim of priority is made upon the provisional application.

This invention relates to a kit for rapid detection of Neospora caninum infections in animals, particulary large commercial animals. Specifically, the present invention describes a diagnostic kit based on a latex agglutination assay to carry out a rapid, on-the-site test to identify N. caninum infections in animals.

### BACKGROUND

N. caninum is an intracellular protozoan parasite and the causative agent of bovine neosporosis, a disease of the reproductive system of cattle as described in “Neosporosis-like abortions in a herd of dairy cattle”, by J P Thilsted and J P Dubey, in the 1989 Veterinary Diagnotic Invest, 1:205-209 which is incorporated herein by reference. Abortion or stillbirth is the only clinical sign of N. caninum infection in cattle. The infection has been associated with sporadic, endemic and epidemic abortions. Pregnant cows of any age may abort from three months of gestation until term. Fetuses may die in utero, be resorbed, mummified, autolyzed, stillborn, born alive but diseased, or born clinically normal but chronically infected. See article “Neosporosis in Cattle”, by M L Anderson et al, 2000 Animal Reproduction Science 60-61: 417-431; “Recent advances in Neospora and neosporosis”, by J P Dubey, 1999 Veterinary Parasitol, 84:349-367 and “A Review of Neospora caninum and neosporosis”, by J P Dubey et al, 1996, Veterinary Parasitol, 67:1-59. These reference articles are incorporated herein by reference.

The disease causes devastating economic losses to the farmers as well as beef and dairy industries. There is no effective vaccine available for the prevention of neosporosis at present. Farmers have to depend on detection and elimination of infected or carrier animals from the herds. The presence of antibodies of N. caninum in an animal indicates that it is, or has recently been, infected with the parasite. Based on this fact, several types of diagnostic test are available commercially at present. These tests utilize either whole parasites or their crude antigen extracts. Several other experimental diagnostic tests are also reported in the literature. See “Serological diagnosis of Neospora caninum infection”, by C. Bjorkman et al, 1999 In. J. Parasitol, 29: 1497-1508 which is incorporated herein by reference. However, there is no rapid detection test available for diagnosing animals with neosporosis. All the presently available serological tests can only be performed in a properly equipped laboratory by appropriately trained personnel. Additionally, the presently available antigen reagents cause the development of high backgrounds, forcing the inclusion of several kinds of control tests and appropriate analysis of the results before judging whether a sample if positive for neosporosis. The present invention describes methods for developing a kit for rapid detection of neosporosis in animals. This test provides clear and unambiguous results and can be performed on the farm or on the site by even untrained personnel.

### SUMMARY OF THE INVENTION

The present invention relates to a diagnostic kit for rapid detection of animals infected with N. caninum. Specifically, this invention relates to the development of a rapid, latex agglutination test utilizing specific recombinant proteins of N. caninum. The invention relies on routine techniques in the field of recombinant DNA technology and immunodiagnostics, well known to those of ordinary skill in the art.

### DETAILED DESCRIPTION OF THE INVENTION

Several proteins of N. caninum to which infected animals develop antibodies have been described in the article of Bjorkman cited previously, and incorporated herein. The gene sequences for these proteins are available in the databases, for instance, such as those of GenBank. These genes are obtained via the polymerase chain reaction or through conventional gene cloning techniques. Each of the specific genes can be cloned, and produced as fusion proteins in Escherichia coli using any of the commercially available expression systems. The expressed N. caninum proteins are then purified according to the specific manufacturer's recommended procedures. For example, the GRA7 protein of N. caninum is produced as polyhistidine-tag fusion in E. coli using the pRSET expression plasmid system ( Invitrogen Inc., Carlsbad, Calif.), and then purified via metal affinity chromatography on nickel-resin. Several of the purified N. caninum proteins can be covalently liked to the surface of latex beads, available in the market (e.g., Bangs Laboratories, Inc., Fishers, Ind. ). The diagnostic kit comprising this invention comprises the prepared latex beads, plastic cards or glass slides, plastic sticks for mixing the solutions, and positive and negative control serum samples.

To perform the test, plasma or serum is collected from the animal and mixed with the prepared latex beads. After incubating the mixture for a few minutes ( typically from 1 to 5 minutes ), the presence of the specific antibody in the sample is detected based on the clumping of the beads which is readily detectable.

Thus, having described the preferred embodiment of the invention, it will be obvious to those of ordinary skill in the art that many changes and modifications can be made without departing from the scope of the appended claims.

1. A diagnostic kit for detecting the presence of N. caninum infection in animals, said kit comprising,
latex beads, slides for analysis, and positive and negative control serum samples.

2. The kit of claim 1 wherein said latex bead have purified N. caninum proteins thereon.

3. The kit of claim 1 and including mixing sticks for mixing serum taken from the animal and the latex beads.

4. A method of diagnosing an animal to ascertain the presence of N. caninum infection therein, said method comprising
taking a serum sample from the animal,
mixing said sample with a solution containing latex beads with purified N. caninum proteins thereon,
comparing solution samples with positive and negative control serum samples to ascertain the presence or absence of infection.

5. The method of claim 4 wherein said comparing step involves analyzing slides with samples of said solution with said control samples on glass slides.

6. The method of claim 4 wherein said purified expressed N. caninum proteins are produced as fusion proteins in Escherichia coli using commercially available expression systems.
